(19) United States
(12) Patent Application Publication
(10) Pub. No.: US 2013/0338488 A1
(43) Pub. Date: Dec. 19, 2013

(54) HIGH FREQUENCY COIL APPARATUS

(71) Applicants: Peter Dietz, Furth (DE); Martin Schramm, Erlangen (DE)

(72) Inventors: Peter Dietz, Furth (DE); Martin Schramm, Erlangen (DE)

(21) Appl. No.: 13/915,697

(22) Filed: Jun. 12, 2013

(30) Foreign Application Priority Data
Jun. 19, 2012 (DE) 102012210280.9

(51) Int. Cl. A61B 5/055

(52) U.S. Cl. CPC A61B 5/0555
USPC 600/415; 600/410

(57) ABSTRACT

A high frequency coil apparatus for a magnetic resonance apparatus and a magnetic resonance apparatus with a high frequency coil apparatus are provided. The high frequency coil apparatus includes a receiving area for receiving a patient and structural elements. Flexible surface elements extend between the rib elements and enclose the receiving area. A high frequency structure is arranged on a structural element. The flexible surface elements are designed to dampen oscillations and to minimize acoustic radiation.

### CROSS REFERENCE TO RELATED APPLICATIONS

The present application claims priority to German Patent Application No. 102012210280.9 DE filed Jun. 19, 2012, the entire content of which is hereby incorporated herein by reference.

### FIELD OF INVENTION

The present invention relates to a high frequency coil apparatus for a magnetic resonance apparatus which comprises a receiving area for receiving a patient, and to a magnetic resonance apparatus having an inventive high frequency coil apparatus.

### BACKGROUND OF INVENTION

Magnetic resonance apparatus have high operating noises during operation, which may have an unpleasant effect on patients who are kept in a receiving area of the magnetic resonance apparatus for examination purposes. Large vibration amplitudes, which result in a significant noise radiation for the patient, appear in particular on a surface of a high frequency coil apparatus which surrounds the receiving area for the patient. Furthermore, acoustic resonances may develop within the receiving area, which may represent an additional noise load. Conventional magnetic resonance apparatuses have a rigid, fixedly installed housing shell unit with a high noise radiation.

The unexamined patent application DE 10 2006 008 724 A1 discloses a magnetic resonance system for instance with a magnet generating a static magnetic field, in the cavity of which an essentially tubular inner cladding is arranged, into which a patient support apparatus can be introduced. With systems of this type, attempts were already made to reduce the noise level in the interior of the tube by means of additional dampening measures on the inner cladding. The additional dampening means nevertheless also reduce the free volume inside the tube.

### SUMMARY OF INVENTION

An object is to provide a high frequency coil apparatus for magnetic resonance apparatus, in which an effective noise protection in a receiving area is achieved for the patient, wherein as large a free volume as possible for the patient is retained at the same time inside of the high frequency coil apparatus. The object is achieved by the features of the independent claims.

An inventive high frequency coil apparatus for a magnetic resonance apparatus comprises a receiving area for receiving a patient, structural elements, which surround the entire exterior of the receiving area, as well as surface elements which extend between the structural elements such that the entire exterior of the receiving area is enclosed by the surface elements. The inventive high frequency coil apparatus further includes a high frequency structure, which is arranged on a structural element. The surface elements are flexible and designed so as to dampen oscillations and minimize acoustic radiation.

The structural elements in this way form a skeleton which encloses the receiving area for the patient. The surface elements extend between the structural elements and seal the entire exterior of the receiving area such that a patient inside the receiving area is separated from the surrounding magnetic resonance device during an examination by means of a magnetic resonance apparatus using an inventive high frequency coil apparatus by means of the surface elements. It is advantageous here for the flexible surface elements to be suited to dampening oscillations and thus to at least partially protect the patient against the noises generated by a scanning process in the magnetic resonance apparatus. At the same time, the required structural rigidity is provided by the structural elements so as to keep the patient in a desired position in the receiving area and to arrange the high frequency structure in a constant position for a measurement. It is advantageous here for the high frequency structure, as a possible oscillation source, to be mechanically separated from the receiving area by means of the oscillation-dampening surface elements, under the influence of the alternating magnetic fields.

The invention also relates to a magnetic resonance apparatus having a magnet unit, which includes a main magnet, a gradient coil and an inventive high frequency coil apparatus.

On account of the features of the inventive high frequency coil apparatus which have already been described, this is able to implement examinations by means of magnetic resonance using a reduced noise load for the patient.

Advantageous embodiments are described in the dependent claims.

In a preferred embodiment of the high frequency coil apparatus, the surface elements form a continuous, closed surface on the side of the surface elements facing the receiving area.

The continuous surface advantageously does not have any edges or interruptions, which contribute to an unwanted sound radiation. Furthermore, it is more easily possible to clean a continuous surface and to comply with hygiene requirements.

In one possible embodiment, the structural elements comprise a cross-section, the dimension of which in the direction parallel to the surface delimiting the receiving area, which is formed by the surface elements, is smaller than the dimension in the vertical direction relative to the surface.

It is advantageous here if the structural elements have a higher rigidity in the direction of the interior of the receiving area and can thus withstand forces in this direction, caused by the weight of the patient and magnetic fields, particularly well. The effective radiation surface is thus simultaneously reduced for sound waves of the structural elements in the direction of the interior of the receiving area.

It is also conceivable in one embodiment for the structural elements to be surrounded by an oscillation-dampening material.

This advantageously dampens oscillations excited directly in the structural elements and furthermore reduces the radiation into the air. The noise level in the receiving area can therefore be further reduced.

In a preferred embodiment, the structural elements and/or the surface elements comprise a lower absorption coefficient for high frequency, particularly in the frequency range between 10 MHz and 150 MHz which is relevant to the magnetic resonance imaging method.

This advantageously ensures that as large a portion as possible of the HF energy radiated by the high frequency coil apparatus reaches the receiving area and likewise as large a portion as possible of the signal emitted by the examination object or the patient again reaches the high frequency structure of the high frequency coil apparatus.

In a preferred embodiment, the structural elements include a carrier element, which extends along the receiving area and rib elements, which extend laterally away from the carrier element and at least partially around the receiving area.

The carrier element can advantageously receive the weight including the patient weight across the length of the receiving area and be dimensioned correspondingly strongly, while the rib elements only distribute the weight and tension forces of the surface elements and can be dimensioned to be accordingly weaker and lighter.

In a preferred embodiment, provision is made for the high frequency coil apparatus also to comprise a patient couch, which is arranged in the receiving area, wherein the carrier element is arranged in a subarea which is separated from the receiving area by the patient couch.

The room which is divided between a patient couch and a conventionally curved wall of the receiving area can on the one hand be used advantageously. It is particularly advantageous here that the patient couch can be arranged directly on the carrier element by reason of the weight to be carried, that by reason of the space available under the couch, it can also be embodied with a correspondingly large cross-section with sufficient rigidity without reducing the space available to the patient in the receiving area.

In a preferred embodiment, provision is also made for the patient couch to be arranged so as to be moveable on the carrier element. This easily enables the patient to be positioned in the receiving area.

### BRIEF DESCRIPTION OF THE DRAWINGS

Further advantages, features and details result from the exemplary embodiments described below and with the aid of the drawings, in which:

FIG. 1 shows a magnetic resonance apparatus with an inventive high frequency coil apparatus in a schematic representation;

FIG. 2 shows a perspective view oblique from above of a first embodiment of a high frequency coil apparatus with elements shown semi-transparently and

FIG. 3 shows a cross-section through an inventive high frequency coil apparatus along the line of intersection III-III in FIG. 2.

### DETAILED DESCRIPTION OF INVENTION

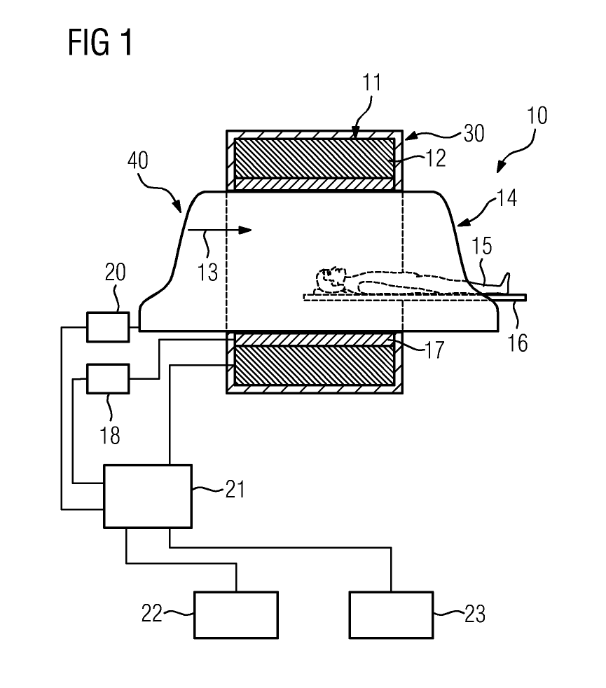

FIG. 1 shows a schematic representation of an inventive magnetic resonance apparatus 10. The magnetic resonance apparatus 10 includes a magnet unit 11 with a main magnet 12 for generating a strong and in particular constant main magnetic field 13. Furthermore, the magnetic resonance apparatus 10 includes a cylindrical receiving area 14 for receiving a patient 15, wherein the receiving area 14 is enclosed in a peripheral direction by the magnet unit 11. The patient 15 can be moved into the receiving area 14 by means of a patient couch 16 of the magnetic resonance apparatus 10. The patient couch 16 is to this end arranged so as to be moveable within the magnetic resonance apparatus 10. Furthermore, the magnetic resonance apparatus 10 comprises a housing unit 30 surrounding the magnet unit 11.

The magnet unit 11 further comprises a gradient coil 17 for generating magnetic field gradients, which is used for local encoding during an imaging process. The gradient coil 17 is controlled by means of a gradient control unit 18. Furthermore, the magnet unit 11 comprises a cylindrical high frequency coil apparatus 40 and a high frequency control unit 20 for exciting a polarization, which is produced in the main magnetic field 13 generated by the main magnet 12. The high frequency coil apparatus 40 is controlled by the high frequency control unit 20 and irradiates high-frequency magnetic resonance sequences into an examination room, which is essentially formed by the receiving area 14. The magnetization is herewith deflected from its position of equilibrium. Furthermore, magnetic resonance signals are received by means of the high frequency coil apparatus 40.

In order to control the main magnet 12, the gradient control unit 18 and to control the high frequency control unit 20, the magnetic resonance apparatus 10 comprises a control unit 21 formed by a computing unit. The computing unit centrally controls the magnetic resonance apparatus 10, such as for instance implementing a predetermined imaging gradient echo sequence. Control information such as for instance imaging parameters, as well as reconstructed magnetic resonance images, can be shown on a display unit 22, for instance at least one monitor, of the magnetic resonance apparatus 10 for an operator. Furthermore, the magnetic resonance apparatus 10 comprises an input unit 23, by means of which information and/or parameters can be input by an operator during a measuring process.

The magnetic resonance apparatus 10 shown can naturally include or if applicable comprise further components which magnetic resonance apparatus 10 usually comprise. The general functionality of a magnetic resonance apparatus 10 is further known to the person skilled in the art, so a detailed description of the general components is omitted.

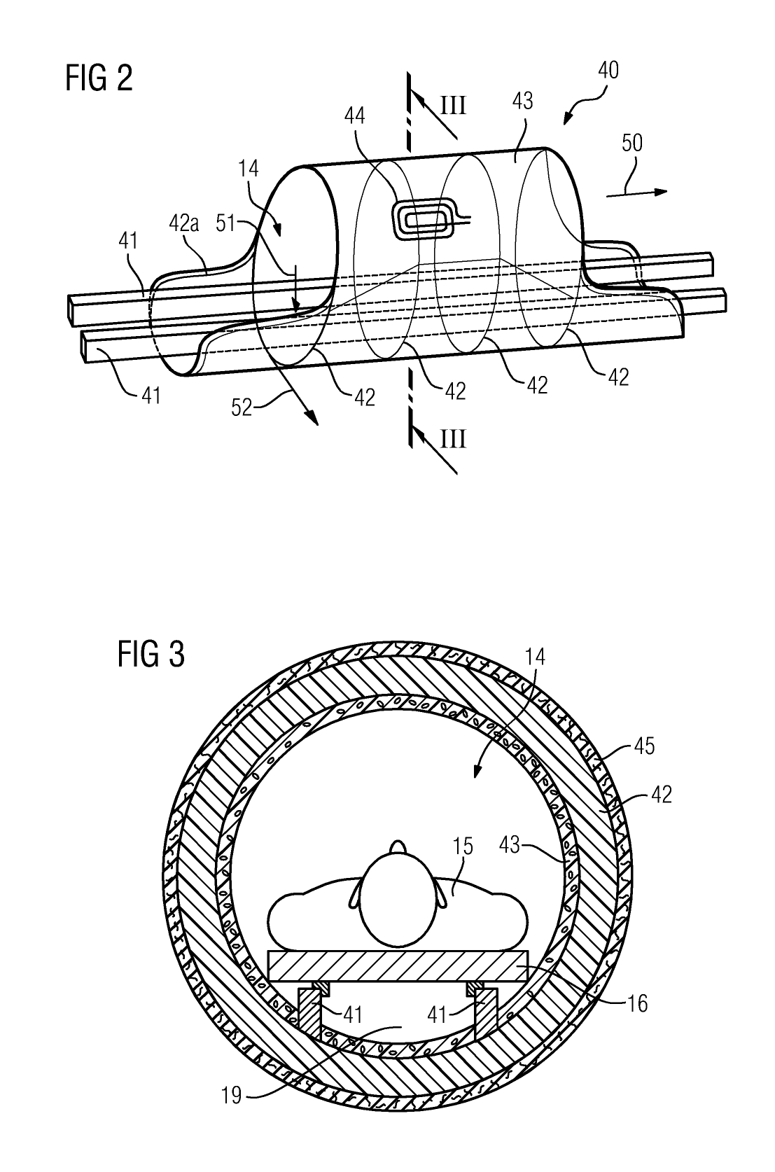

FIG. 2 shows an inventive high frequency coil apparatus 40 in a perspective view, such as is used inventively in a magnetic resonance apparatus in FIG. 1. In this case individual elements 43 are shown semi-transparently so as to render structures 42 hidden there behind as visible.

A preferred embodiment of the inventive high frequency coil apparatus 40 comprises at least one carrier element 41, as well as a number of rib elements 42 as structural elements 41, 42.

The embodiment shown in FIG. 2 inter alia includes two carrier elements 41. The carrier elements 41 are oriented in an axial direction, which corresponds to the longitudinal orientation of the receiving area 14. If the high frequency coil apparatus 40 is located in a magnetic resonance apparatus 10, this axial direction 50 is thus aligned in the direction of the main magnetic field 13. Deviations from this alignment are however also conceivable.

The carrier elements 41 comprise a cross-section at right angles to the axial direction, which, in a first direction, is larger in a plane at right angles to the axial direction than in a second direction in this plane. If the high frequency coil apparatus 40 is in a magnetic resonance apparatus 10, the first direction is thus essentially aligned in the radial direction 51 and parallel to the direction of gravity, so that the carrier element comprises a greater rigidity for gravitational forces and radially active forces than for tangential forces in the second direction essentially parallel to the tangential direction 52. The carrier elements therefore deform less under their own weight and the weight of a patient 16, at the same time the surface of the carrier element 41 is smaller which is aligned to the receiving area and is available for sound radiation purposes.

The rib elements 42 extend laterally from the carrier elements 41. In the embodiment shown, the rib elements 42 extend in the manner of an arc about the cylindrical receiving area 14. It is however also conceivable for the receiving area to have another cross-section, such as for instance an ellipse or a polygon and for the rib elements to have a corresponding shape. It is also conceivable for the rib elements 42 not to have a closed shape, but instead only to enclose part of the receiving area 14. Furthermore, it is conceivable for the rib elements 42a to also extend in an axial direction 50, for instance for better access to the peripheral ends of the receiving area.

Aside from the embodiment shown, other forms and arrangements of structural elements are also conceivable, for instance grids made of triangles, rhombi, polygons or honeycomb structures, which surround the entire surface of the receiving area 14.

The rib elements 42 can be fastened laterally in the tangential direction 52 or also on the side of the carrier elements 41 facing away from the receiving area 14 using conventional fastening means such as glue, screws, rivets or suchlike. It is also conceivable for the carrier and rib elements to be produced in one piece.

In one embodiment, the rib elements 42 also comprise a cross-section at right angles to the direction of extension, which is larger in the radial direction 51 than in the axial direction 50. As a result, the surface of the rib elements 42 is less, which is directed at the receiving area 14 and available for sound radiation purposes. At the same time the rigidity is also increased for radial stresses.

This asymmetric cross-section is also conceivable in other embodiments with other structural elements.

Flexible surface elements 43 are arranged between the rib elements 42 and/or the carrier elements 41. The surface elements 43 are in this way arranged such that they enclose the entire exterior of the receiving area 14, so that the rib elements 42 and/or the carrier elements 41 are not exposed in the receiving area 14 and no sound can be directly radiated there.

The surface elements 43 can be produced from various sound-dampening materials such as elastic foam material or others. It is preferable here for the surface elements 43 to have smooth surfaces, since these are easier to clean and more easily comply with hygiene requirements. It would also be conceivable for the surface elements 43 to be provided with an additional smooth surface made of another material.

The surface elements 43 can be configured such that they only cover areas between individual rib elements 42 or also a number of rib elements 42 at the same time. Provision is made here for transition elements to be provided at the abutting edges between individual surface elements, which allow a continuous surface to be created without abutting edges. A surface element 43 may therefore comprise a lug in each instance, which runs along the abutting edge, protrudes beyond an adjacent surface element 43 and is fastened there to a fastening means, for instance a Velcro fastener. The closed surface facilitates cleaning and minimizes sound radiation.

In one embodiment, the surface elements 43 can be fastened to the rib elements 42 and/or the carrier elements 41 using various fastening means. It is also conceivable for the surface elements 43 to be glued. It is also conceivable however for mechanical fastening elements such as lugs, screws, Velcro fasteners or suchlike to be used. It is furthermore conceivable, with a suitable choice of the shape of the surface element 43 and rib element 42, that these are kept in engagement with the rib element 42 by means of a force and/or form closure solely by a reset force of the flexible surface element.

A high frequency structure 44 is arranged on the structural elements 41, 42. The high frequency structure 44 may comprise various forms, such as for instance saddle coils, or also cylindrical coils, which surround the entire exterior of the receiving area. The high frequency structure 44 can be arranged for instance on just on structural element 41, 42 or extend across a number of structural elements 41, 42. The structure comprising rib elements 42 and carrier elements 41 forms a rigid framework, which enables a precise and unchangeable position in respect of the receiving area 14 for the high frequency structure 44. The structure comprising rib elements 42 and carrier elements 41 is also able to receive forces which are exerted on the high frequency structure 44 by alternating magnetic fields. Since these forces can also cause oscillations in the rib elements 42 and the carrier elements 41, it is advantageous for the rib elements 42 and/or carrier elements 41 not to be exposed in the receiving area 14 and thus not to contribute directly to the sound radiation. The surface elements with their sound-absorbing consistency dampen on the one hand the oscillations of the structural elements 41, 42, but nevertheless simultaneously only transmits the sound into the receiving area 14 to a minimal degree.

As shown in the cross-section in FIG. 3, the rib elements 42 and/or the carrier elements 41 can further be provided with an oscillation-dampening coating 45. This may be a plastic foam for instance with corresponding dampening properties.

In the preferred embodiment in FIG. 3, a patient couch 16 can be arranged in the receiving area 14. The carrier elements 41 are to this end arranged in parallel to one another in the longitudinal direction of the receiving area 14 in a lower subarea 19 of the receiving area 14. The patient couch 16 and/or the carrier elements 41 comprise transport means 46, which allow for a movement of the patient couch 16 into and out of the receiving area 14. To this end, the top side of the carrier element 41 can be embodied in the manner of rails so that wheels on the underside of the patient couch 16 allow for a rolling of the couch, wherein a lateral guidance is simultaneously ensured. Other transport means such as runners, axial ball bearings or suchlike are however also conceivable.

Although the invention was illustrated and described in more detail by the preferred exemplary embodiment, the invention is not restricted by the disclosed examples and other variations can be derived here from by the person skilled in the art without departing from the protective scope of the invention.

1. A high frequency coil apparatus for a magnetic resonance apparatus, the high frequency coil apparatus comprising:
a receiving area for receiving a patient,
structural elements which surround an entire exterior of the receiving area,
surface elements which extend between the structural elements such that the entire exterior of the receiving area is enclosed by the surface elements, and
a high frequency structure which is arranged on one of the structural elements,
wherein the surface elements are flexible and are designed to dampen oscillations and to minimize acoustic radiation.

2. The high frequency coil apparatus as claimed in claim 1, wherein the surface elements form a continuous, closed surface on a side facing the receiving area.

3. The high frequency coil apparatus as claimed in claim 1,
wherein the structural elements have a cross-section,
wherein a dimension of the cross-section in direction parallel to a surface delimiting the receiving area, said surface being formed by the surface elements, is smaller than a dimension in a vertical direction in respect of the surface.

4. The high frequency coil apparatus as claimed in claim 1, wherein the structural elements are surrounded by an oscillation-dampening material.

5. The high frequency coil apparatus as claimed in claim 1, wherein the structural elements and/or the surface elements comprise a low absorption coefficient for high frequency.

6. The high frequency coil apparatus as claimed in claim 1, wherein the structural elements comprise
a carrier element which extends along the receiving area and
rib elements which extend laterally away from the carrier element and at least partially around the receiving area.

7. The high frequency coil apparatus as claimed in claim 6,
wherein the high frequency coil apparatus further comprises a patient couch which is arranged in the receiving area,
wherein the carrier element is arranged in a subarea separated from the receiving area by the patient couch.

8. The high frequency coil apparatus as claimed in claim 7, wherein the patient couch is arranged so as to be moveable on the carrier element.

9. A magnetic resonance apparatus, comprising:
a magnet unit which includes a main magnet, a gradient coil and a high frequency coil apparatus as claimed in claim 1.
